(19) United States
(12) Patent Application Publication
(10) Pub. No.: US 2023/0013142 A1
(43) Pub. Date: Jan. 19, 2023

(54) IODINE COMPOUNDS FOR TREATING RESPIRATORY PATHOGENS

(71) Applicant: IOCURE, INC, Lawrence, NY (US)

(72) Inventor: Mark Daniel FARB, Lawrence, NY (US)

(21) Appl. No.: 17/821,895

(22) Filed: Aug. 24, 2022

Related U.S. Application Data
(63) Continuation-in-part of application No. PCT/US2021/023574, filed on Mar. 23, 2021.
(60) Provisional application No. 62/993,085, filed on Mar. 23, 2020.
(60) Provisional application No. 63/026,122, filed on May 17, 2020.

(30) Foreign Application Priority Data
Jul. 7, 2020 (IL) 275909

(51) Int. Cl. A61K 33/18; A61P 31/14; A61K 9/00; A61K 45/06

(52) U.S. Cl. CPC A61K 33/18; A61P 31/14; A61K 9/0073; A61K 45/06

(57) ABSTRACT

Provided herein are compositions, methods, uses, and articles of manufacture for iodine treatment on mucosal membranes, and treatment of respiratory pathogens in this way—e.g., by inhalation and combined with the evaporation of steam. In certain embodiments, iodine treatment encompasses administration of compounds that release molecular iodine and/or physiologically active iodine-containing compounds.

### FIELD OF THE INVENTION

Disclosed herein are methods for treating respiratory infections, comprising use of a heated iodine and iodide salt solution.

### BACKGROUND

There are many respiratory viruses, the most recent serious one being current coronavirus (COVID-19). Influenza is widespread and killed 60,000 people in the US in one recent winter. There are other hard-to-treat pathogens, such as SARS, respiratory syncytial virus, resistant tuberculosis, antibiotic resistant pneumonias, candidiasis, and more.

Viruses are a large problem because the antibiotics for them are rare and not very effective.

There are respiratory bacterial pathogens that do not respond well to antibiotics. As one example, the treatment of tuberculosis is meeting increasing drug resistance.

In summary, there is a need for a broadly effective antibiotic useful against many kinds of respiratory system pathogens, particularly viruses.

### Iodine and Iodine Compounds in Medicine

Iodine solution is a known antiseptic for skin, for aqueous solutions, and for air sterilization.

Iodine compounds have other medical uses. Potassium iodine has long been used for thyroid conditions and to block radioactive iodine uptake in nuclear emergencies.

The overwhelming consensus of doctors is that iodine is unsafe for other uses. Almost every doctor has seen in standard textbooks such as Harrison's Principles of Medicine and Goodman and Gilman's pharmacology texts that iodine is toxic above a certain level for inspiration, and there is an additional perception that iodine is unsafe for mucosal surfaces, as death can occur from excessive gastrointestinal exposure to iodine. PubChem (https://pubchem.ncbi.nlm.nih.gov/compound/Iodine#section=NIPH-Clinical-Trails-Search-of-Japan) only lists an antibiotic use of iodine on the skin.

There are also commercial gargles using iodine (Eggers et al. 2018, In Vitro Bactericidal and Virucidal Efficacy of Povidone-Iodine Gargle/Mouthwash Against Respiratory and Oral Tract Pathogens. Infect Dis Ther. 2018 June; 7(2): 249-259) and potassium iodide has been used as lavage fluid (Derscheid et al., Am J Respir Cell Mol Biol. 2014 February; 50(2): 389-397. Increased Concentration of Iodide in Airway Secretions Is Associated with Reduced Respiratory Syncytial Virus Disease Severity.)—all at room temperature.

Scientific American's June 2020 issue featured COVID-19. An article (Waldholz) on fast-track drugs does not even consider the use of iodine or any treatment other than complex antiviral drugs and various immunological treatments. That reflects state-of-the-art thinking about how to deal with severe viral respiratory infections.

When the applicant wrote an email to a pulmonologist at a major medical center about using iodine for his COVID patients, he responded with an article downloaded from Fisher Scientific about the toxicity of iodine. It contains the phrase “Harmful if inhaled”!

### SUMMARY OF THE INVENTION

Here is a summary of the aspects that will be discussed in more detail. The applicant is leveraging the properties of iodine chemistry in conjunction with information from diverse and unconnected medical specialties and environmental biology to innovate a way of treating difficult respiratory infections.

a. The antibiotic agent is in the form of an aqueous vapor. The applicant found no reference in the literature of using aqueous iodine vapor to treat infections.

b. The vapor is heated. Since heating a substance is generally regarded to increase its activity and toxicity, there is no reference in the literature to using a heated aqueous vapor of iodine in treatment.

c. In some embodiments, the solution forming the vapor contains not only iodine but also an iodine-releasing salt. (“Salt” by definition includes substances such as Povidone which are organic but ionize in water.) Some compounds containing dissolved iodine release it into solution. This is the first time that the nature of such compounds to form equilibrium mixtures in water has been leveraged to deliver active elemental iodine, which is what reacts with pathogens, in a much safer form. The small percentage of elemental iodine reacts with the pathogen in the respiratory tract, and immediately more is drawn out of the solution to restore equilibrium. In other embodiments, the solution forming the vapor contains pure iodine.

d. The upper temperature is limited (typically to 80 deg. C.) so that not too much elemental iodine is released. Below a certain temperature, there is minimal activity. This is a safety precaution based on applying equilibrium and heated solution effects to the current problem.

e. The parts per million can be increased beyond the limits thought possible, because those limits were based on pure iodine vapor, whereas, in solution accompanied by a salt, generally at least 90% of the iodine is dissolved in salt form.

f. The total iodine dosage per day can be capped below toxic levels, and the total allowed amount per day can be concentrated into a smaller amount of time that is more reasonable for treating a patient in a potentially critical situation because the chemistry allows the use of a higher concentration. (The possibilities for toxicity are both from the concentration of free elemental iodine at any one time and the total amount allowed per day; the new method of delivery enables a solution.)

g. Iodine is more effective against pathogens at a higher pH. That alkalinity can be delivered as part of the aqueous vapor and was not relevant when pure elemental iodine vapor was used. The alkalinity can also be used to reduce the dose and thereby the toxicity.

The applicant challenges the perception that iodine is unsafe on mucosal surfaces from some little-known ophthalmic research on the successful and safe use of substances such as Povidone iodine drops for conjunctival sterilization prior to eye surgery. (One reference is Isenberg and Apt. The Ocular Application of Povidone-Iodine, Community Eye Health. 2003; 16(46): 30-31.) In addition, the applicant, an ophthalmologist, occasionally prescribed Povidone drops for patients with methicillin-resistant Staphylococcus Aureus eye infections; he received calls in all cases from nurses, pharmacists, or doctors like “Dr. Farb, do you know what you are doing?” After they received literature references and proceeded, the applicant had a 100% success rate in treating those infections. Since most pulmonologists and infectious disease experts do not read the ophthalmic literature, they would find such an idea dangerous, as typified by the referenced email.

The next question is how to deal with iodine's known respiratory toxicity.

The applicant disputes the prevailing medical practice by using another discipline, physical chemistry of the halogens, which virtually all physicians are unaware of. Iodine-releasing ionic compounds such as Povidone and potassium iodide (as opposed to substances containing iodine such as thyroxine, wherein the iodine is bound to other atoms and not capable of being released into salt form without breaking the bonds) exist in an equilibrium in a solution with elemental (also termed molecular) iodine. Here is the key formula for an example of potassium iodide, a likely source of the iodine treatment and the ingredient in Lugol's Solution:

KI(aq)+I₂(s)→KI₃(aq)

KI₃ is highly soluble, therefore only small amounts of I₂ are available while in solution. That reduces the toxicity from inspiration. When the I₂ reacts with pathogens, the equilibrium draws out the creation of more I₂ from KI₃. Because the iodine-releasing compounds are highly soluble, there is little free iodine present in these solutions, maybe 1-10% according to various studies.

Another example is hypoiodous acid, HIO. It rapidly decomposes: 5 HIO→HIO₃+2 I₂+2 H₂O.

Since free iodine is what reacts with and inactivates pathogens, and most body cells can handle it (iodine in solution is required for basic health and circulates in the blood to be used in the thyroid gland), providing iodine in heated and inspired solution to the respiratory tract would be a way to apply it so it would have little toxic effect until it lands on a surface and attacks pathogens.

Warming such an iodide salt solution would have additional benefits of making it easier to reach the lungs and anywhere else in the respiratory system, and of making it more reactive for the first few seconds until it reaches body temperature.

Steaming an aqueous solution of iodine-releasing compound that forms an equilibrium with a small amount of free iodine, as far as the applicant knows, has never been considered before, likely because of the difficulty bridging the disciplines of infectious disease and pulmonary medicine, ophthalmology, pharmacology, and physical chemistry, and the simultaneous fear of iodine being painful, toxic, and messy.

Without wishing to be bound by theory, iodine in association with warm, moist air elicits a more vigorous reaction from the higher temperature and loosening of any layers of material, whether the product of human or pathogenic metabolism, deposited on the lung surface, thereby enabling greater therapeutic efficacy. It has been reported that COVID-19 emits a coating on the lung's mucosal surface. This method of treatment would help to penetrate that layer.

### Dosage and Toxicity

An alternative is the use of higher than supposedly safe doses because short pulses at high parts per millions can kill the virus in emergency situations, and the published toxicity standards are made for prolonged exposure. If one were to use the traditional way of looking at iodine, with dosage according to number of iodine molecules or weight of iodine in the solution, the treatment proposed here would appear to be more toxic than it really is. The reason is that so little elemental iodine is present—and even that is partially retained in solution—that there is little toxicity in the passage through the respiratory tract until it lands on the infected surfaces.

For comparison, the recommended dose of iodine for hyperthyroidism is 750 mg/day. Recommended dose to prevent iodine uptake in the presence of radioactive iodine: 130 mg/day for an adult (CDC). Normal nutrition is 0.150 mg/day. According to Poisoning & Drug Overdose, 6e, 2012, Kent R. Olson, page 1499, short term exposure can be irritating at as low as 0.1 ppm and work is difficult at 1.5-2 ppm. The ACGIH-recommended workplace ceiling limit (TLV-C) for iodine vapor is 0.1 ppm (1 mg/m3). The air level considered immediately dangerous to life or health (IDLH) is 2 ppm. (Owen, Kelly, Poisoning and Drug Overdose, Access Medicine [McGraw Hill Medical]. Chapter 84, Iodine, by Kelly P. Owen) The Burnet and Stone research (see below) suggests complete inactivation of viruses within 1 minute in vitro at 0.1 ppm. Decreased virulence of viruses occurred at 0.005 ppm.

The CDC website (https://www.cdc.gov/nceh/radiation/emergencies/ki.htm) states as follows: “According to the FDA, the following doses are appropriate to take after internal contamination with (or likely internal contamination with) radioactive iodine: Newborns from birth to 1 month of age should be given 16 mg (¼ of a 65 mg tablet or ¼ mL of solution). This dose is for both nursing and non-nursing newborn infants. Infants and children between 1 month and 3 years of age should take 32 mg (½ of a 65 mg tablet OR ½ mL of solution). This dose is for both nursing and non-nursing infants and children. Children between 3 and 18 years of age should take 65 mg (one 65 mg tablet OR 1 mL of solution). Children who are adult size (greater than or equal to 150 pounds) should take the full adult dose, regardless of their age. Adults should take 130 mg (one 130 mg tablet OR two 65 mg tablets OR two mL of solution). Women who are breastfeeding should take the adult dose of 130 mg.”

A toxicity study for the EU (https://echa.europa.eu/registration-dossier/-/registered-dossier/5883/7/6/2) showed data for an experiment on rats ingesting liquid potassium iodide for a 2-year period. A short bottom line is that 10 ppm showed no long-term effects, and only 100 ppm showed a long-term decrease in life span.

In some embodiments, the concentration of iodine will be lower in vapor than in solution. In certain embodiments, e g to avoid toxicity, the resulting air mixture iodine concentration in the lungs or respirator input tubes is less than 2 ppm (pure iodine vapor concentration) for extended-period dosing. In other embodiments, this is calculated based on, e.g., the equilibrium concentration of iodine in the solution and the temperature and the flow rate of the input air. In yet other embodiments, pulses of higher than 2 ppm respirator air iodine are used, since Bennett and Stone found virus kill rates superior at higher concentrations than 2 ppm. In this manner, kill rate can be increased by the temporary use of a higher dose. Such a higher dose may be, in some embodiments, above a daily toxicity level if it were continuously administered for an entire day.

WHO Guidelines for Drinking-water Quality, 1996, 2nd ed. Vol. 2, states as follows: “Doses of 30-250 ml of tincture of iodine (about 16-130 mg of total iodine per kg of body weight) have been reported to be fatal. Acute oral toxicity is primarily due to irritation of the gastrointestinal tract, marked fluid loss and shock occurring in severe cases.”

Yeon and Jung (Yeon and Jung, Effects of temperature and solution composition on evaporation of iodine as a part of estimating volatility of iodine under gamma irradiation, Nuclear Engineering and Technology, Volume 49, Issue 8, December 2017, Pages 1689-1695.) performed I₂ evaporation experiments with I₂ and I-mixed solutions in the temperature range 26-80° C. in an open, well-ventilated space. The evaporation of I₂ was observed to follow primarily first order kinetics, depending on the I₂ concentration. The evaporation rate constant increased rapidly with increase in temperature. Their FIG. 4 shows that evaporation at 50 degrees Centigrade is over 10 times more rapid than at 26 degrees, room temperature; and evaporation at 80 degrees is over 30 times faster than at 26 degrees.

In light of the above information, and the fact that iodine vapor readily penetrates the vascular system from the lungs, it is reasonable to be cautious and take the lowest of the above toxicity measures into account as a maximum, that is, 16 mg/kg/day. For a margin of safety, we suggest that the total iodine content be no greater than 8 or 10 mg/kg/day. As a result, the combination of not exceeding 2 ppm pure iodine concentration in vapor (for other than short pulses) maximum and 16 mg/kg/day would keep the patient from the lowest levels of generally recognized toxicity, and that in practice making the maximums 10 mg/kg/day and 0.1 ppm would clearly be safe. Even with respirator use, it is likely that planning on a 1 ppm level would mean that much would be lost through the air, so there is a built-in safety factor for both the lung dose and the dose absorbed into the blood. Therefore, the applied dosage is likely to be effective at 10% or less of the potentially toxic dose.

Let us take a case with Lugol's iodine. Using 0.3 ml of Lugol's solution per minute at 50 degrees and a delivery rate of 6 liters of air per minute (the average), a solution concentration of 0.1 ppm will result in 248 minutes of treatment in order to deliver the maximum non-toxic dose per day for an average adult. Raising the concentration to 1 ppm and increasing cadence and quantity of breaths to 12 liters of air per minute results in a treatment time of 12 minutes to reach the maximum non-toxic dose for the day, If the doctor were to decide that a patient could take up to 250 mg of iodide per day, and if that were to be delivered over a short period of time at 1 ppm, it would take several minutes to deliver. In certain embodiments, to enable levels of iodine that are safe but have anti-pathogenic activity, the ventilator or other iodine delivery device (which may be any device described herein) calculates the total number of milligrams per kilogram (mg/kg) per day—based on the volume of air introduced to the patient and the concentration of the iodine in total and/or in bioactive form—such that the total never goes above 16 mg/kg per day, and that it can be set to lower and safer levels such as (in various embodiments) 15, 10, 8, 5, or 1 mg/kg per day, with the ability to customize, in some embodiments, the total amount administered for patients with conditions such as thyroid disease. The ventilator or other iodine delivery device can also be set to make a time-based calculation, so that the patient would, for example, receive 15 mg/kg or less in the course of one hour, once per day, to enable a more concentrated regimen of iodine with a higher number of ppm than would be possible if the patient received a maintenance dose steadily throughout 24 hours. There is a basis for that in the Bennett and Stone in vitro studies of a greater viral kill rate from higher concentrations. This is summarized in FIG. 2.

### In Vitro

The literature on the effectiveness of iodine on viruses in a solution is almost entirely on cold iodine solutions and is in all cases outside the human body, for the purpose of sanitizing the air or water.

As reported in Letters in Applied Microbiology (51(2):158-63), Andreas Sauerbrei and Peter Wutzler studied successful use within 5 minutes of iodine against a variety of viruses on the skin.

In 1945, Stone and Burnet (Stone and Burnet, The Action Of Halogens On Influenza Virus With Special Reference To The Action Of Iodine Vapour On Virus Mists, Australian Journal of Experimental Biology and Medical Science, 1 Sep. 1945; https://onlinelibray.wiley.com/doi/abs/10.1038/icb.1945.32) created a bin in which a virus mist was exposed to iodine vapor. The experiment involved the placement of iodine crystals in those bins to produce the vapor. Another way they produced the vapor involved dissolving iodine crystals in methanol. They found that 0.1 parts per million destroyed the influenza viruses. Below that, there was a destructive effect but not total. This was the effect of a mist on a mist, not an in vivo infection. In all cases, they introduced the iodine in the test chamber before introducing the mice (not a typical therapeutic scenario). They were interested in air sterilization. They give the practical suggestion to impregnate gauze masks for doctors and nurses with iodine. They published a related article as Burnet et al. (Burnet et al., Action of iodine vapour on influenza virus in droplet suspension, Aust J Sci. 1945 February; 7:125) This approach was not tested in humans and was used in prevention and antisepsis with non-infected mice, instead of treatment. Iodine was neither vaporized with steam nor nebulized.

FIG. 1 is a table from Stone et al. on the effective concentrations for virus kills in the outside air (not in the lungs). The inventor proposes that the concentration of iodine in the inspired air should ideally be in the range of 0.01-0.2 ppm based on this data, but it should be safe to go to 2 ppm, in certain embodiments. However, the chemistry of iodine solutions would enable higher ppm of iodine in air droplets. The inventor suggests that short pulses of relatively higher concentrations of iodine (e.g., 0.1 ppm and higher) would have a faster therapeutic effect without risking toxicity rather than a lower concentration for a longer time. This can be, in certain embodiments, adjusted by a control mechanism, in some embodiments computerized, coupled with a timer in the ventilator or other iodine delivery device.

Eggers 2019, Infectious Disease Management and Control with Povidone Iodine, Maren, Infectious Diseases and Therapy volume 8, pages 581-593(2019)) states: “Following application, elemental iodine can take on several forms in aqueous solution, with the molecular I₂ and hypoiodous acid (HOI) being the most effective in terms of antimicrobial activity. The iodine molecules are free to oxidise vital pathogen structures such as amino acids, nucleic acids and membrane components. An equilibrium is achieved in such circumstances, with more PVP-bound iodine released into solution to replace the iodine that is consumed by germicidal activity. The maintenance of this equilibrium ensures long-lasting efficacy during bouts of microorganism proliferation, as well as better tolerability for patients due to lower levels of irritation. Electron microscopy and biochemical observations support the hypothesis that PVP-I disrupts microbial cell walls by inducing pore formation, leading to cytosol leakage. The lack of reported resistance to PVP-I to date is thought to be due to the sheer diversity of susceptible targets within each pathogen, an important aspect to be considered in the face of rising concerns for antibiotic resistance.” They continue, “PVP-1 refers to an iodine preparation commonly used in both household and healthcare settings. It consists of a complex of povidone, hydrogen iodide, and elemental iodine which targets structures critical to the survival and replication of microorganisms. Common formulations typically consist of a 10% PVP-I solution containing 1% available iodine.”

The above studies illustrate how the amount of potentially toxic iodine is limited in solution and is safer than previous medical practice allows.

pH

In still other embodiments, pH is adjusted to improve effectiveness of iodine. Hsu and Nomura (Hsu and Nomura, Sterilization Action Of Chlorine And Iodine On Bacteria And Viruses In Water Systems (US Army Technical Report)) found in Experiment 10 that higher pH resulted in a higher kill rate. In certain embodiments, substances that can lend a higher pH to the iodine/vapor combination, without it becoming caustic to the lungs, constitute a method and formulation to enhance the treatment's effectiveness. Gomez et al., proposed that inhalation of aerosol of a bicarbonate solution, resulting in a higher pH between 7 and 8 in most cases, reduced sputum viscosity in cystic fibrosis. (Gomez et al., Safety, Tolerability, and Effects of Sodium Bicarbonate Inhalation in Cystic Fibrosis. Clinical Drug Investigation, Nov. 13, 2019). Another formulation could include carbonate or hydroxide.

Eschenbacher W L, Gross K B, Muench S P, Chan T L (Eschenbacher W L, Gross K B, Muench S P, Chan T L, Am Rev Respir Dis. 1991 February; 143(2):341-5. Inhalation of an alkaline aerosol by subjects with mild asthma does not result in bronchoconstriction) https://www.ncbi.nlm.nih.gov/pubmed/1990950) found that asthma patients had no reactive vasoconstriction with alkaline vapor as high as pH 10.3. This means that an alkaline solution or vapor with iodine up to pH 10.3 would be safe and likely more effective than iodine alone.

### Definitions

The respiratory system consists of the mouth, nose, sinuses, pharynx (upper respiratory system); and trachea and lungs (lower respiratory system). Most respiratory pathogens affect both, but the infection in the lungs is usually more serious. Infections in the trachea and/or lungs are referred to herein as a lower respiratory infection, and such infections represent an embodiment of a disease treated by the described methods, compositions, uses, and articles of manufacture.

Influenzas and especially coronaviruses affect or penetrate the mucosal lining of the lung in their most serious form. Given the inventor's belief in the safety and efficacy of iodine against viruses on mucus membranes, an additional question is how to get it into the lungs.

A warmed solution is defined as a solution above 25 degrees ° C. (room temperature), which results, in certain embodiments, in release of therapeutically effective amounts of volatile iodine (e.g., at 26° C. or higher)—or, in other embodiments, within another temperature range mentioned herein, each of which represents a separate embodiment. Realistically, heating to at least 30 degrees substantially improves the delivery and reactivity.

### BRIEF DESCRIPTION OF THE DRAWINGS

FIG. 1 is a table from Stone et al. on the effective iodine concentrations for kills of Influenza in a mist.

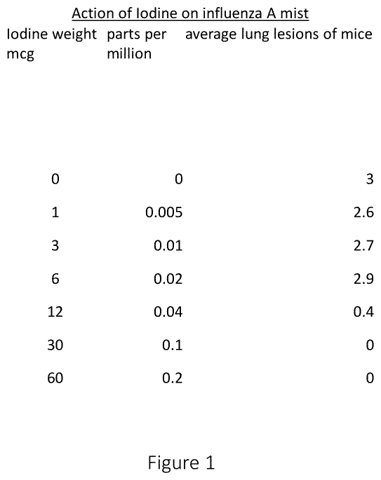

FIG. 2 is a schematic depiction of exemplary, non-limiting inputs 200 for calculating a dose of inhaled iodine, including, for example, target exposure time 201, dose 202, pH 203, respirator cadence and/or pressure 204, fluid amount 205, concentration 206, fluid temperature 207, patient weight 208, and air flow rate 209. Inputs are used to program CPU 220 and which in turn calculates target milligrams of iodine inspired per time 221 and instructs control mechanisms 230, including, for example, addition of iodine to system 231 (e.g., via addition of iodine or an iodine-releasing compound to solution in the device, or via supplying additional iodine-containing solution to the device), iodine pulsing 232, pH 233, and respirator cadence and/or pressure 234. This figure is not meant to limit the number of inputs. As discussed herein, the expected volume of the patient's respiration could be a factor used in calculating the ppm over a period of time. It is possible that the computer may be programmed with a default calculation of the patient's breath volume based on age, weight, or height, or it may rely on the volume of the actions of the respirator or other factors.

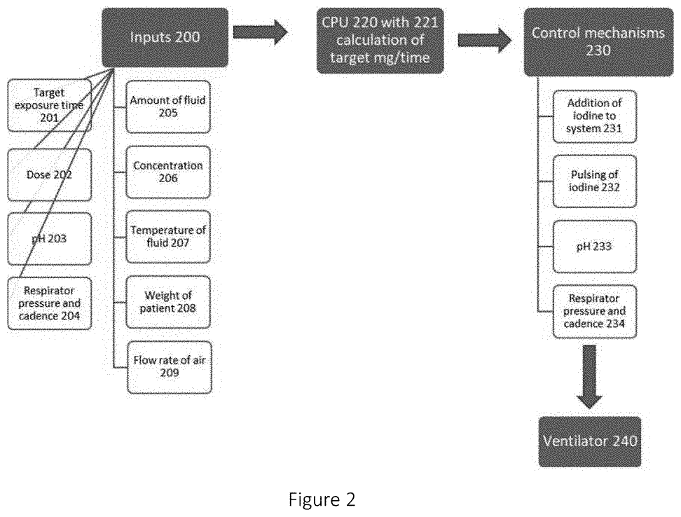

FIG. 3 is a schematic of an exemplary iodine, alkali, or other formulation autofill process in conjunction with a respirator. A computer 301 with memory receiving input from at least one sensor 302 instructs a first actuator 303 to open a first valve 304 to release therapeutic material (not shown) from a reserve container 305 to a vaporization container (306), from which there is a connection via respiratory tubing 307 to the patient's respiratory system (not shown). That container is also connected via control by computer 301 to a downstream actuator 308 and second valve (309) in order to open or block the communication to the respiratory system. This computer control relies on at least one sensor 302, which may be a volume sensor, whereby the volume of therapeutic substance or equivalent measure is taken in order to determine proper dosing and proper movement through the first valve 304. In the case of alkali autofill, or a combination of alkali and iodine, for example, there may be an optional pH sensor. The most important is to sense volume so it is clear when the vaporization container 305 needs to be refilled.

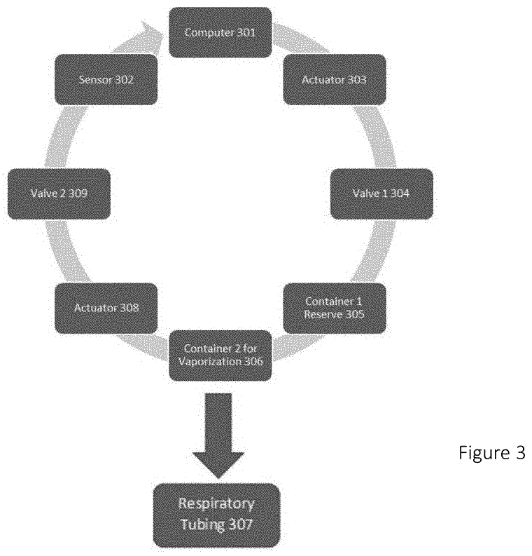

FIG. 4 is a graph of temperature and oxygen saturation of patients treated with inhaled iodine.

### DETAILED DESCRIPTION

Provided herein are methods, compositions, uses, and articles of manufacture for treating respiratory tract infections, comprising iodine and compounds that generate iodine and/or pharmacologically active iodine species.

In some embodiments, there is provided a composition, comprising a vapor, for administration to a respiratory tract of a subject infected with a respiratory pathogen, wherein: (a) the vapor is generated by warming an aqueous solution having a pH of 7.0-10.0, the solution comprising an iodine-releasing ionic compound and elemental iodine in a total iodine concentration of 1.5-10 ppm, to a temperature of 30-80 degrees centigrade (° C.), thereby generating the vapor; (b); the administration is for 2 hours or less per day; and (c) total iodine delivered to the patient's respiratory tract does not exceed 8 mg/kg of body weight/day. In certain embodiments, the vapor comprises elemental iodine. In other embodiments, the vapor further comprises an iodine-releasing ionic compound. It is clarified that the specified pH range refers to the final pH of the solution that is heated to produce the vapor administered to the patient.

In other embodiments, there is provided a system for treating a subject infected with a respiratory pathogen, the system comprising: (a) a container with an aqueous solution having a pH of 7.0-10.0 disposed therein, the aqueous solution comprising an iodine-releasing ionic compound and elemental iodine in a total iodine concentration of 1.5-10 ppm; (b) a heating element configured for warming the aqueous solution in the container to a temperature of 30-110 degrees centigrade (° C.), thereby generating a vapor; and (c) a ventilator configured to ventilate the vapor such that the vapor reaches a respiratory tract of a subject infected with a respiratory pathogen; wherein the ventilator is configured for administration of the vapor for 2 hours or less per day, wherein total iodine delivered to the respiratory tract does not exceed 8 mg/kg of body weight/day. In certain embodiments, the vapor comprises elemental iodine. In other embodiments, the vapor further comprises an iodine-releasing ionic compound.

In other embodiments, there is provided a measured dosage pack, comprising elemental iodine and an iodine-releasing ionic compound, accompanied by instructions for (a) introducing the dosage pack into an aqueous solution, having a pH of 7.0-10.0, in a total iodine concentration of 1.5-10 ppm; (b) warming the aqueous solution to a temperature of 30-110 degrees centigrade (° C.), thereby generating a vapor; and (c) administering the vapor to a respiratory tract of a subject infected with a respiratory pathogen, wherein the administration is for 2 hours or less per day, wherein total iodine delivered to the respiratory tract does not exceed 8 mg/kg of body weight/day. In certain embodiments, the vapor comprises elemental iodine. In other embodiments, the vapor further comprises an iodine-releasing ionic compound.

In various embodiments of methods, systems, and compositions described herein, the vapor is in the form of droplets and/or comprises elemental iodine and/or the described iodine-releasing ionic compound.

In other embodiments, there is provided a composition comprising a vapor or nebulized liquid, the vapor or nebulized liquid comprising elemental iodine or an iodine-releasing compound, for administration to a lower respiratory tract of a subject infected with a respiratory pathogen (e.g., a pathogen infecting the respiratory tract).

In other embodiments, there is provided a method of treating a subject infected with a lower respiratory pathogen, comprising administering to the subject's lower respiratory tract a vapor or nebulized liquid, the vapor or nebulized liquid comprising elemental iodine and/or an iodine-releasing compound. In other embodiments, the iodine-containing liquid is warmed to generate a vapor, and the vapor is subsequently or simultaneously nebulized.

In yet other embodiments, there is provided use of a vapor or nebulized liquid comprising elemental iodine and/or an iodine-releasing compound, in the manufacture of a medicament for treating a respiratory pathogen via administration to a lower respiratory tract of a subject.

In other embodiments, there is a provided a measured dosage pack, in liquid or solid form, comprising elemental iodine or an iodine-releasing compound. The use of saturated potassium iodide solution (commercially available as SSKI®) is one standard in use and is described herein. One embodiment as an example would involve diluting 1 ml of SSKI by a factor of 10. Then 1 ml would contain 76.4 mg of iodine, which is well within the safe dosage for a day. That 1 ml could be vaporized to the patient's lungs over a sequence of time chosen by the doctor. Then, in another embodiment, solely for exemplification purposes, that amount could be timed for delivery so that the parts per million in the volume of inspired air would be 0.1 ppm, Since reports of discomfort according to NIOSH start at 1.5 ppm, it should be safe for a limited time to set the concentration to 1 ppm in order to have a stronger virus kill that is well within the limits of safety. In more extreme cases of danger to the patient from the respiratory infection, the doctor could decide to increase the concentration, for an example, up to 1.9 or other target ppm on a time-limited basis such as 10 minutes. The dosage pack is indicated for introduction into a solution, after which the solution is warmed, thereby producing a vapor. The vapor is indicated for administration to a lower respiratory tract of a subject infected with a respiratory pathogen. In certain embodiments, the solution is warmed prior to introduction of the iodine or iodine-releasing compound. In other embodiments, the solution is warmed subsequent to introduction of the iodine or iodine-releasing compound into the solution.

In other embodiments, there is a provided a measured dosage pack, comprising elemental iodine or an iodine-releasing compound. The dosage pack is indicated for introduction into a solution, after which the solution is nebulized or warmed, thereby producing droplets. The droplets are indicated for administration to a lower respiratory tract of a subject infected with a respiratory pathogen. In other embodiments, the iodine-containing solution is warmed to generate a vapor, and the vapor is subsequently or simultaneously nebulized.

In yet other embodiments, there is provided an article of manufacture, comprising (a) a measured dosage pack comprising elemental iodine or an iodine-releasing compound; and (b) a label comprising instructions for (i) combining contents of the measured dosage pack with a solution; (ii) warming the solution to a temperature above 25° C., to produce a vapor; and (iii) administering the vapor to a subject infected with a respiratory pathogen.

In still other embodiments, there is provided an article of manufacture, comprising (a) a ventilator, operably connected with a liquid reservoir and a solution comprising elemental iodine or an iodine-releasing compound; and (b) a label comprising instructions for (i) warming the solution, thereby generating a vapor; and (ii) administering the vapor to the lower respiratory tract of a subject infected with a respiratory pathogen. Those skilled in the art will appreciate, in light of the present disclosure that, typically, the reservoir may be configured to warm a solution contained therein (e.g., it may be associated with a heating element).

In still other embodiments, there is provided an article of manufacture, comprising (a) a ventilator, operably connected with a nebulizer and a solution comprising elemental iodine or an iodine-releasing compound, wherein the nebulizer is configured to nebulize the solution into droplets; and (b) a label comprising instructions for (i) nebulizing the solution into droplets; and (ii) administering the droplets to the lower respiratory tract of a subject infected with a respiratory pathogen. In other embodiments, the iodine-containing liquid is warmed to generate a vapor, which is disposed within the ventilator, and the vapor is subsequently nebulized with another device associated with, or in other embodiments disposed within, the ventilator. In other embodiments, the iodine-containing liquid is warmed to generate a vapor, which is disposed within the ventilator, and the vapor is simultaneously nebulized with another device associated with, or in other embodiments disposed within, the ventilator.

The described systems and articles of manufacture comprising, in some embodiments, a powder or a vial to be put into a specific amount or temperature range of water. In other embodiments, a measured dosage pack, ampule, or liquid formulation for patient use includes temperature specifications in the instructions. Amounts of iodine inspired from a given solution would vary with the temperature of the solution at the time of administration, and the therapeutic level would be reached more easily at higher temperatures.

In still other embodiments, there is provided a measured dosage pack, comprising elemental iodine or an iodine-releasing compound, indicated for introduction of the dosage pack into a warmed solution, wherein the warmed solution is disposed in a ventilator, thereby producing a vapor for administration to a subject infected with a respiratory pathogen).

In yet other embodiments, there is provided a method of treating a subject infected with a respiratory pathogen, comprising administering to said subject a heated vapor, comprising elemental iodine or an iodine-releasing compound. In other embodiments, there is provided a composition for treating a respiratory pathogen, comprising a heated vapor, said heated vapor comprising elemental iodine or an iodine-releasing compound. In still other embodiments, there is provided use of a heated vapor comprising elemental iodine or an iodine-releasing compound, in the manufacture of a medicament for treating a respiratory pathogen. In other embodiments, there is provided an article of manufacture, comprising (a) a vaporizable liquid comprising elemental iodine or an iodine-releasing compound; and (b) a packaging material, wherein the packaging material comprises a label instructing a user to treat subject infected with a respiratory pathogen, via heating said vaporizable liquid to generate a vapor and administering said vapor to said subject.

In some embodiments, the solution (into which the contents of the measured dosage pack are introduced) is disposed in a ventilator. In other embodiments, the solution is disposed within an evaporation device operably connected with the described ventilator, e.g., so that the vapor produced by the solution is introduced into the ventilator.

In other embodiments, the solution is disposed in an outpatient device, which may be, in certain embodiments, a non-invasive breathing assistance apparatus, e.g., a breathing tube or respiratory face mask. In other embodiments, the outpatient device is a room humidifier, household pot, or any other type of evaporation-facilitating device that does not require a professional to operate.

In still other embodiments, there is provided an article of manufacture, comprising (a) a measured dosage pack, comprising elemental iodine or an iodine-releasing compound; and (b) a packaging material, comprising a label instructing a user to: (i) introduce the dosage pack into a solution; (ii) warm the solution; and (iii) administer a vapor of the warmed solution to a lower respiratory tract of a subject, in order to treat a respiratory pathogen.

In still other embodiments, there is provided an article of manufacture, comprising (a) a pharmaceutical composition comprising a vaporizable liquid, the vaporizable liquid comprising elemental iodine or an iodine-releasing compound; and (b) a packaging material, comprising a label instructing a user to use the composition in treating a respiratory pathogen, via administration of a vapor of the liquid to a lower respiratory tract of a subject.

The described dosage packs and/or compositions are, in certain embodiments, accompanied by instructions regarding their frequency of use, non-limiting embodiments of which are once-daily dosages and twice-daily dosages and continuous dosages for a specified period of time. Such instructions enable, in some embodiments, control of the daily dosage to which the subject is exposed.

Iodine, Iodine-Releasing Ionic Compounds, and Solutions Comprising Same

The term “iodine” is used herein to refer to the element itself, e.g., in its common molecular form “I subscript 2”. Iodine-releasing compounds encompass iodine-releasing salts, such as hypoiodous acid; and ionic molecules containing and releasing iodine. Solutions containing iodine and iodine-releasing compounds are also encompassed in the described methods, compositions, uses, and articles of manufacture. In certain embodiments, the described compounds are able to generate bioavailable iodine (I sub 2) for the purpose of vaporization. In certain embodiments, iodine is present in the described vapor in a therapeutically effective amount, that is, an amount having anti-microbial activity. In other embodiments, iodine is present in any of the amounts or ranges mentioned herein.

The described iodine-releasing compounds (of any method, composition, use, or article of manufacture mentioned herein) include, in certain embodiments, compounds that release free elemental iodine or another active iodine compound in solution or vapor. The term is not intended to encompass compounds that do not release free elemental iodine or another active iodine compound in solution or vapor, even if they comprise iodine atoms—non-limiting examples of such compounds are raw seaweed and thyroid hormone. In other embodiments, the iodine-releasing compound is a volatile compound.

In certain embodiments, the iodine-releasing compound for vaporization has a boiling point below 200° C. (or, in other embodiments, below 150° C., 125° C., or 100° C.); or, in other embodiments, a room-temperature half-life in aqueous solution of at least 30 minutes (or, in other embodiments, at least 45 minutes, 60 minutes, 90 minutes, or 120 minutes); or, in other embodiments, both characteristics, which may be freely combined. Those skilled in the art will also appreciate, considering the present disclosure, that the particular active iodine species is not critical for reducing to practice the described methods and compositions. In certain embodiments, whatever the active iodine species, the described solutions and/or vapors contain elemental iodine at an appreciable concentration, which is, in certain embodiments, any of the concentrations mentioned herein.

In other embodiments, the described iodine-releasing compound (of any method, composition, use, or article of manufacture mentioned herein) is hypoiodous acid (HOI), povidone iodine (2-Pyrrolidinone, 1-ethenyl-homopolymer), an organic or inorganic iodine carrier, or an iodine salt, each of which represents a separate embodiment. In certain embodiments, the iodine salt is potassium iodide, sodium iodide, or a mixture thereof.

It will be appreciated by those skilled in the art, in light of the present disclosure, that the term “active iodine compound(s)” refers to iodine-containing compounds with significant anti-viral activity at concentrations achievable by the described devices. In other embodiments, e.g., in the case of vaporized compositions, additional advantageous characteristics are sufficient lability to be vaporized at effective concentrations and sufficient stability to survive the journey from the device to the subject's respiratory tract.

Solely by way of exemplification, 0.1 ppm of iodine=1.038 mg/m³. Potassium iodide can be conveniently prepared as a saturated solution, abbreviated SSKI. SSKI contains about 764 mg iodide per mL. There are around 15 drops per mL; the iodide dose is therefore approximately 51 mg per drop. Therefore, to achieve 0.1 ppm in a cubic meter of air, one drop of SSKI should be added to 50 drops of water.

The uncomplexed molecular iodine (I₂) is, in some embodiments, the active ingredient in the described iodine solution. This is the amount that could be used for dosage calculation, and for the method of determining the amount of biocidal uncomplexed iodine in ventilators, combined with the understanding that a new equilibrium is continuously reached. The concentration of other compounds containing iodine can also be measured, but the dosage calculation based on I₂ is used, in certain embodiments, for greater ease of standardization. Such calculations are known to those skilled in the art; non-limiting examples of them are provided in Wadai et al. (Wada et al., Relationship between Virucidal Efficacy and Free Iodine: Concentration of Povidone-Iodine in Buffer Solution, Biocontrol Science, 2016, Vol. 21, No. 1, 21-270).

In yet other embodiments, the described solution (of any method, composition, use, or article of manufacture mentioned herein) comprises both elemental iodine and an iodine-releasing ionic compound (non-limiting examples of which are povidone iodine, potassium iodide, sodium iodide, and a combination thereof). As provided herein, the presence of an iodine salt increases the solubility of elemental iodine in aqueous solutions. Further, the presence of both elemental iodine and an iodine salt (e.g., in equilibrium) enables replenishing of elemental iodine levels, enabling maintenance of elemental iodine levels within a therapeutic range over the course of many hours.

In certain embodiments, as the iodine solution is heated and molecular iodine and/or other bioactive iodine compounds evaporate with the steam, the molecular iodine in the solution is replenished by reaching a new equilibrium with the salts or carriers still in solution.

The iodine-containing solution of the described method, composition, use, or article of manufacture contains, in some embodiments, molecular iodine at a solution concentration of 0.1-10 parts per million (ppm); or, in other embodiments, 0.2-10 ppm, 0.3-10 ppm, 0.4-10 ppm, 0.5-10 ppm, 0.6-20 ppm, 0.8-20 ppm, 1-20 ppm, 0.2-15 ppm, 0.3-15 ppm, 0.4-15 ppm, 0.5-15 ppm, 0.6-15 ppm, 0.8-15 ppm, 1-15 ppm, 0.2-10 ppm, 0.3-10 ppm, 0.4-10 ppm, 0.5-10 ppm, 0.6-10 ppm, 0.8-10 ppm, 1-10 ppm, 1-10 ppm, 2-10 ppm, 3-10 ppm, 4-10 ppm, or 5-10 ppm. In certain embodiments, the dosage is calibrated such that the iodine content in the vapor administered in one day is less than 8 mg/kg of body weight of the subject.

The iodine-containing vapor of the described method, composition, use, or article of manufacture contains, in some embodiments, molecular iodine at a vapor concentration of 0.01-2 parts per million (ppm); or, in other embodiments, 0.02-2 ppm, 0.03-2 ppm, 0.04-2 ppm, 0.05-2 ppm, 0.06-2 ppm, 0.08-2 ppm, 0.1-2 ppm, 0.02-1.5 ppm, 0.03-1.5 ppm, 0.04-1.5 ppm, 0.05-1.5 ppm, 0.06-1.5 ppm, 0.08-1.5 ppm, 0.1-1.5 ppm, 0.02-1 ppm, 0.03-1 ppm, 0.04-1 ppm, 0.05-1 ppm, 0.06-1 ppm, 0.08-1 ppm, 0.1-1 ppm, 0.1.5-1 ppm, 0.2-1 ppm, 0.3-1 ppm, 0.4-1 ppm, or 0.5-1 ppm. In certain embodiments, the dosage is calibrated such that the iodine content in the vapor administered in one day is less than 8 mg/kg of body weight of the subject.

In yet other embodiments, e.g., for an exposure time of 24 hours or less, molecular iodine is present in the described vapor at a concentration over 1.5 ppm, over 2 ppm, over 3 ppm, over 4 ppm, or over 5 ppm. In other embodiments, iodine is present in the solution at a concentration over 15 ppm, over 20 ppm, over 30 ppm, over 40 ppm, or over 50 ppm. The exposure time of the subject to the vapor is, in various embodiments, less than 20 hours, less than 16 hours, less than 12 hours, less than 10 hours, less than 8 hours, less than 6 hours, less than 4 hours, less than 3 hours, or less than 2 hours. The aforementioned iodine concentrations and exposure times may be freely combined. In still other embodiments, a vapor containing very high concentrations of iodine, e.g., 2-10 ppm, 3-10 ppm, 4-10 ppm, 5-10 ppm, 2-5 ppm, or 2-4 ppm is administered to a subject for 30-60 minutes; for 15-30 minutes; or for 1-15 minutes. In certain embodiments, the treatment may be administered up to twice daily.

In other embodiments, e.g., for a subject with thyroid disease, molecular iodine is present in the solution at a concentration of 0.1-5 ppm; or, in other embodiments, 0.2-5 ppm, 0.3-5 ppm, 0.4-5 ppm, 0.5-5 ppm, 0.6-5 ppm, 0.8-5 ppm, or 1-5 ppm.

In certain embodiments, the described iodine-containing vapor is produced by warming a solution or mixture comprising iodine (and/or, in various embodiments, an iodine-containing compound) to a temperature above 25 degrees centigrade (° C.). In more specific embodiments, the solution or mixture is warmed to 26-80° C., 30-80° C., 35-80° C., 40-80° C., 45-80° C., 50-80° C., 55-80° C., 60-80° C., 65-80° C., 70-80° C., 75-80° C., 80-80° C., 26-100° C., 30-100° C., 35-100° C., 40-100° C., 45-100° C., 50-100° C., 55-100° C., 60-100° C., 65-100° C., 70-100° C., 75-100° C., or 80-100° C.

In certain embodiments, the described vapor has been obtained from a solution that has a pH between 7.0-10.3, between 7.0-10.0, between 7.0-9.5, between 7.0-9.0, between 7.5-10.3, between 7.5-10.0, between 7.5-9.5, or between 7.5-9.0.

In other embodiments, the described vapor, when allowed to condense, has a pH between 7.0-10.3, between 7.0-10.0, between 7.0-9.5, between 7.0-9.0, between 7.5-10.3, between 7.5-10.0, between 7.5-9.5, or between 7.5-9.0.

Iodine Delivery Systems

In various embodiments, all systems of providing air (including oxygen), vapor, or medication to a subject's respiratory system are encompassed as means of delivering iodine and iodine-releasing compounds. That includes for example ventilators, humidifiers, vaporizers, and both specialized and non-specialized heating containers (e.g., a kitchen pot) that can produce warmed or steaming vapor. The terms “ventilator” is intended to encompass any type of apparatus that physically contains and influences the composition of the air inspired by a subject. In certain embodiments, the described ventilator is closed, or, in other embodiments, at least partially closed. In other embodiments, the ventilator comprises a breathing tube. Enclosed respirator systems, e.g., PAPR (powered air-purifying respirators), are included. An Ambu bag (Bag valve mask) is considered another embodiment of a ventilator. “Face masks” refer to masks enabling respiration of inspired air, with or without medication, and are also considered, in another embodiment, a component of ventilators (protective face masks are not encompassed). Nasal prongs for air inhalation, usually supplementary oxygen, are also, in another embodiment, a component of ventilators. In certain embodiments, the described ventilator is a mechanical ventilator, defined as a mechanized device that enables the delivery or movement of air and/or oxygen into the lungs of a patient whose breathing has ceased, is failing, or is inadequate. In further embodiments, the mechanized ventilator has any of the following attributes (alone or in combination): a. monitors and customizes gas delivery; b. maintains a minimal pressure in the lungs (e.g., to prevent the alveoli from collapsing), and c. delivers air and/or oxygen to the lungs by way of a tube inserted into the trachea through the mouth or nose.

The inventor proposes that the treatment will be better in those ventilators that use heated wires to maintain water vapor saturation and warm temperatures, because there will be less water vapor condensation (and thus iodine condensation in the tubes or upper respiratory system), thus enabling the iodine to reach the lungs. In the same way, if a patient is breathing from a vaporizer or a pot of heated water with iodine added, he should inhale close to the source to make sure more iodine reaches the lungs. Deep breaths should be encouraged in all cases, and particularly coronavirus, which causes a coating on the lung surface, so that peripheral areas of the lung dependent on mucociliary clearance obtain a greater benefit from the treatment. The inventor's recommendation is to make sure the ventilator is set to maximize iodine exposure to the lung periphery. Since it is important that the iodine reaches the periphery of the lungs, the option of adjusting the pressure and periods of inhalation (cadence) to reach the lung periphery should be available as part of the control system advocated by the applicant.

Due to some reports (Swift, Diana, Medscape News, Apr. 13, 2020, Higher Mortality Rate in Ventilated COVID-19 Patients in Large Sample (Medscape, article 928605) that excessive pressure from respirators may increase morbidity from coronavirus, it is suggested that, in some embodiments, such use of increased pressure be used only for short time periods coinciding with increased dosing of the iodine.

In some embodiments, the ventilator, humidifier, vaporizer, or heating container of the described method, composition, use, or article of manufacture comprises an iodine autofill system. Alternatively or in addition, the ventilator or other iodine delivery device comprises an alkali autofill system, which, in other embodiments, is configured to maintain a target pH range of the solution contained therein, which may be, in various embodiments, any pH or pH range mentioned herein. In certain embodiments, that alkali can be bicarbonate or a bicarbonate salt.

In certain embodiments, the iodine autofill system helps maintain exposure to the lungs over an extended period. The level of total iodine administration (in both free and salt form) can be set by an autofill controller and an apparatus comprising a computer control system with memory that releases a set amount of compound, held in a container communicating with the ventilator and equipped with a valve controlled by the computer, during a particular time range. This can be combined, in certain embodiments, with a sensor to detect the concentration of iodine, and the sensor sends data to the computer, which then sends instructions to the valve controller. In other embodiments, a pH sensor is included to monitor the pH of the ventilator fluid and have an autofill operating in a similar fashion for an alkali such as sodium bicarbonate to maintain the pH at a particular number. The computer can be set to compile data of the amount dosed over time in order to keep the total iodine amount dosed below toxicity level and provide it via a user interface, by wireless or cable communication with the computer, and to generate alerts locally and via the computer. An input device or interface can be attached to the autofill to customize the regimen for a patient's weight and other conditions.

The described vapor is, in various embodiments, disposed within a breathing tube, a respiratory face mask, or nasal prongs, each of which represents a separate embodiment. In still other embodiments, the vapor is disposed within a ventilator. In yet other embodiments, the vapor is disposed within a humidifier or vaporizer.

Alternatively or in addition, the ventilator, humidifier, vaporizer, or heating container comprises a heating element (non-limiting examples of which are heated wire(s) or other immersed element, a heated plate [which is, in some embodiments, adjacent to the chamber holding the solution], and an element surrounding the chamber holding the solution) that facilitates vaporization of water.

Target Pathogens

The pathogen treated by any of the mentioned methods, compositions, uses, or articles of manufacture, is, in some embodiments, a virus. In more specific embodiments, the virus is COVID-19.

In other embodiments, the coronavirus described herein is human and bat severe acute respiratory syndrome coronavirus (SARS-CoV) of the type severe acute respiratory syndrome-related coronavirus, e.g. SARS-CoV-1 and SARS-CoV-2. In certain embodiments, the treated virus is SARS-CoV-2.

In still other embodiments, the virus is a coronavirus, an influenza virus, a respiratory syncytial virus, a vaccinia virus, a bovine viral diarrhea virus, a polyomavirus SV40, an adenovirus, a mumps virus, a rotavirus, a coxsackievirus, a rhinovirus, a herpes simplex virus, rubella, measles, or a poliovirus, each of which represents a separate embodiment. In other embodiments, the pathogen is another viral pathogen, each of which represents a separate embodiment. In other embodiments, the virus is a lipid-enveloped virus; while in other embodiments, the virus is not lipid enveloped. Alternatively or in addition, the virus expresses a haemagglutinin, a neuraminidase, or both.

In certain embodiments, the target pathogen expresses haemagglutinin. Eggers 2019 states “The influenza virus has been responsible for some of the most significant epidemics in the modern world, with annual outbreaks resulting in approximately 3-5 million cases of severe illness and between 250,000 and 500,000 deaths per year. An influenza study using plaque inhibition assays showed that a 1.56-mg/ml PVP-I treatment can inhibit infections in MDCK cells by human (eight strains) and avian (five strains) influenza A viruses, including H1N1, H3N2, H5N3 and H9N2, from 23 to 98%. Receptor binding analysis revealed that haemagglutinin inhibition was the likely cause of the PVP-I virucidal activity, rather than the inhibition of host-specific sialic acid receptors. The finding also demonstrates two specific mechanisms of reduction of viral growth, namely, PVP-I blockade of viral attachment to the host cell receptors and the inhibition of viral release from infected cells.”

Alternatively or in addition, the target pathogen expresses neuraminidase. Eggers 2019 states “PVP-I formulations are also known to have broad antiviral properties. These effects are mechanistically similar in principle to iodine's antibacterial activity. For example, the virucidal mechanisms of action of PVP-I have been determined to involve the inhibition of essential viral enzymes such as neuraminidase. The inactivation of this enzyme blocks viral release from the host cell, preventing further spread of the virus to uninfected cells. In addition, PVP-I also inhibits viral haemagglutinin, resulting in the blockade of attachment to host cell receptors. By simultaneously targeting both critical aspects of the viral machinery needed for replication, PVP-I reduces the likelihood of resistance emerging through sudden mutation.”

In yet other embodiments, the respiratory pathogen is a bacterial pathogen. In more specific embodiments, the pathogen is tuberculosis, which is, in some embodiments, antibiotic-resistant tuberculosis. In other embodiments, the pathogen is a pneumonia-causing antibiotic resistant bacterial strain. In still other embodiments, the pathogen is another bacterial pathogen, each of which represents a separate embodiment. In other embodiments, it may be any of the bacteria that cause respirator-induced pneumonia. In other embodiments, the pathogen is a fungus, a non-limiting example of which is Candida (e.g., Candida albicans, which is known to cause pneumonia). (Dermawan et al., Mandanas)

Also provided herein is a method for reducing an incidence of pneumonia induced by a ventilator, by administering elemental iodine and an iodine-releasing compound to a subject using steamed vapor in a ventilator, at a dose that does not exceed 16 mg/kg/day of elemental iodine). When added to the input solution, iodine and iodine-releasing compounds can treat and impede development of ventilation-associated pneumonia. According to the US Center for Disease Control, Ventilator-associated pneumonia is a lung infection that develops in a person who is on a ventilator. A ventilator is a machine that is used to help a patient breathe by giving oxygen through a tube placed in a patient's mouth or nose, or through a hole in the front of the neck. An infection may occur if germs enter through the tube and get into the patient's lungs.” A research article they refer to, Klompas M et al., has no mention of iodine.

### Possible Mechanisms of Action

In certain embodiments, without wishing to be limited by theory, the described methods and compositions exert an effect by modification of surface proteins and/or fatty acids. McDonnell and Russell write, “Similar to chlorine, the antimicrobial action of iodine is rapid, even at low concentrations, but the exact mode of action is unknown. Iodine rapidly penetrates into microorganisms and attacks key groups of proteins (in particular the free-sulfur amino acids cysteine and methionine), nucleotides, and fatty acids, which culminates in cell death. Less is known about the antiviral action of iodine, but nonlipid viruses and parvoviruses are less sensitive than lipid enveloped viruses. Similar to bacteria, it is likely that iodine attacks the surface proteins of enveloped viruses, but they may also destabilize membrane fatty acids by reacting with unsaturated carbon bonds.”

Additional Agents

In another embodiment, heparin or another anti-clotting agent is co-administered. One value of the use of iodine, particularly in patients with slow blood clotting times, is to minimize the use of anti-clotting medications in this complication of corona.

Currently, there is much concern about coronavirus causing blood clots, so it is of interest that the iodine reduces hemagglutination in vitro. Sriwilaijaroen (Sriwilaijaroen et al., Mechanisms of the action of povidone-iodine against human and avian influenza A viruses: its effects on hemagglutination and sialidase activities, Virology Journal volume 6, Article number: 124 (2009)) states: “Receptor binding inhibition and hemagglutinin inhibition assays indicated that PVP-I affected viral hemagglutinin rather than host-specific sialic acid receptors.” According to the research cited, the agglutination is related to the virus more than the host.

In certain embodiments, ACE inhibitors (Rohan), vitamin D (McCall), ivermectin, corticosteroids, of which one example is dexamethasone (Giardina), mouthwash (Vlessides)—particularly those with high content of germicidal compound(s) such as alcohol, and/or steroids or other anti-viral compounds are co-administered, each of which represents a separate embodiment. In certain embodiments, the additional compound is administered in the same composition as the iodine or iodine-releasing compound.

In other embodiments, there is provided use of iodine vapor for antisepsis of plant surfaces.

Administration with Drugs Targeting Intracellular pH or Lysosomal pH

In yet other embodiments, the described method, composition, use, or article of manufacture further comprises or utilizes an additional active agent that increases intracellular pH or lysosomal pH. In some, the drug is chloroquine or hydroxychloroquine. Other non-limiting examples of active agents that increases intracellular pH or lysosomal pH are aminoquinolines, for example 4-aminoquinolines, such as amodiaquine, hydroxychloroquine (HCQ), chloroquine; 8-aminoquinolines, such as primaquine and pamaquine; and mefloquine. In certain embodiments, the additional active agent is administered in the same composition as the iodine or iodine-releasing compound.

The inventor suggests combining the iodine treatment with other drugs such as chloroquine, which may inhibit the pathogens in complementary ways, for example, by increasing intracellular pH or pH of endosomes or lysosomes of target cells of the pathogen in the lower respiratory tract. Krogstad and Schlesinger (Am J Trop Med Hyg. 1987 March; 36(2):213-20, The basis of antimalarial action: non-weak base effects of chloroquine on acid vesicle pH.) write, “Biologically active concentrations of chloroquine increase the pH of the parasite's acid vesicles within 3-5 min.” Alternatively or in addition, a pH-raising agent is included in an iodine solution. The present disclosure encompasses each of these embodiments as a new drug combination, whatever the route of administration.

In yet other embodiments, iodine solution with an added pH-raising agent is used concurrently with oral chloroquine treatment.

### Subjects

In certain embodiments, the subject treated by the described compositions, methods, uses and articles of manufacture is a human. In other embodiments, the subject is an animal, non-limiting examples of which are dogs, cats, horses, and cows.

In other embodiments, there is a provided an in vivo use of iodine (or, in other embodiments, an iodine-releasing compound), for impeding formation of blood clots formed in a subject having an infectious disease (e.g., a lower respiratory pathogen), in certain embodiments a coronavirus infection. In certain embodiments, the iodine is administered via inhalation. In other embodiments, the iodine is administered orally. In still other embodiments, another route of administration is utilized, a non-limiting example of which is intravenous administration. In various embodiments, the described anticoagulant use is prophylactic or therapeutic. Alternatively or in addition, the total level of iodine administered is kept below toxic levels, e.g., (for an average patient without complicating diseases) less than 16 mg/kg/day.

### EXPERIMENTAL DETAILS SECTION

### Example 1: Case Study

The inventor suffered from a coronavirus infection and cured himself with inhaled iodine vapor, as detailed in the following diary entries:

On the evening of Monday, March 16, I came back to my Long Island apartment from a day in Manhattan passing through Penn Station (after a previous week of flying from Tel Aviv to Amsterdam, then Amsterdam to JFK, and taking trains within the Netherlands). In the Netherlands I sat next to, for an extended time, a co-worker who became very sick a few days later. Feeling OK.

Tuesday: March 17: Started to get what I thought was a cold.

Wed: March 18-Friday March 20: I started to realize that I didn't just have a cold, as this wasn't following my usual cold symptoms. I started to get fevers and sweats in spite of taking Tylenol every 4 hours until Sunday morning March 22, a huge degree of exhaustion (needing to go back to sleep after being up around 30-60 minutes), a moderate cough, moderate shortness of breath (for me, that meant doing a low intensity 20-minute workout instead of a high intensity 1-2 hour daily workout, and feeling exhausted and short of breath). I also noticed that my ability to taste food had decreased.

Friday, March 20: I realized I had corona, and started to think through my medical experience and knowledge about a cure.

Sunday, March 22, 8:30 AM: I was sweating with fever and could barely make it to my car. I brought with me a vaporizer with water, a lighter to AC electrical converter, and put on a mask. I went to the local pharmacy to buy liquid iodine (both tincture and Povidone). I parked my car, put iodine in the vaporizer (around one fluid ounce in around a quart of water), and breathed from the vaporizer with the windows mostly shut for an hour. I took a break, refilled the vaporizer, and repeated the process. By 12:00, I was feeling 95% better, walking around my apartment and working, just needed a small nap, No fever, no need for Tylenol. I've been doing fine since. I did supplement with some 5-minute breathing sessions over a pot with some water and some iodine after that but didn't really need it.

Several weeks later I had a positive antibody test for corona and two negative nasal swab tests for corona.

### Example 2: Inhaled Iodine Improves Temperature and Oxygen Saturation in Patents with Covid-19

### Methods

To establish the safety profile of the intervention, all patients in the studies to follow were screened extensively before and after entry into the study, using a large battery of tests. No adverse events were reported by any of the indications.

Table 1 illustrates the natural course of the disease in the same location, in nearly age-matched individuals not rigorously given the iodine doses exemplified herein (first cohort). Visit 1 is immediately before treatment, Visit 2 at 3 hours, Visit 3 at 6 hours, Visit 4 at 24 hours, Visit 5 at 1 week. It is clear that there is a general decline without adequate treatment.

TABLE 1
Natural course of COVID-19 infection.

| | Visit 1 | Visit 2 | Visit 3 | Visit 4 | Visit 5 |
|---|---|---|---|---|---|
| temp decline F. | 0 | −0.3 | −0.6 | −0.7 | −0.8 |
| oxygen increase | 0 | −0.7 | −0.6 | −0.8 | −1.1 |
| average temp F. | 100.6 | 100.8 | 101.1 | 101.2 | 101.4 |
| average O2 | 94.9 | 94.3 | 94.4 | 94.2 | 93.9 |

20 patients, 18 males and 2 females, average age 34, formed the second cohort.

The lowest reported fatal dose of iodide compounds is 16 mg/kg/day (800 mg for a 50 kg person), so the dosage was kept substantially below that. Patients received a single dose of 30 ml of Povidone Iodine 10% in 500 ml of water at 50 degrees Centigrade, inhaled for 15 minutes, face partially masked, with constant supervision.

### Results

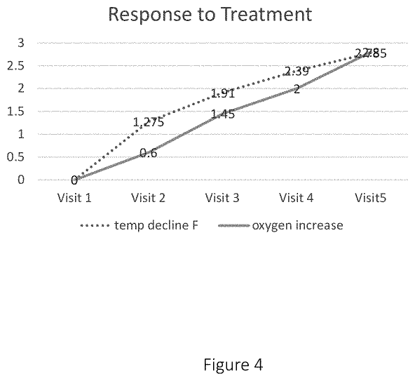

This table reports temperature and oxygen saturation. Significant response was noted within 3 hours, and improvement continued throughout the week. See Table 2 and FIG. 4.

TABLE 2
Response to iodine vapor in cohort 2.

| | Visit 1 | Visit 2 | Visit 3 | Visit 4 | Visit 5 |
|---|---|---|---|---|---|
| | Start | 3 hours | 6 hours | 1 day | 7 days |
| Temp decline F. | 0 | 1.275 | 1.91 | 2.39 | 2.785 |
| Oxygen increase | 0 | 0.6 | 1.45 | 2 | 2.8 |
| Average temp F. | 100.5 | 99.2 | 98.6 | 98.1 | 97.7 |
| Average oxygen | 95.4 | 96 | 96.9 | 97.4 | 98.2 |
| saturation | | | | | |
| (fingertips) | | | | | |

The results indicate substantial improvement in oxygen and temperature within 3 hours that continued steadily throughout the week. All 20 patients responded.

Another 22 patients (cohort 3) were treated with the same regimen but with the solution at pH 8. The results are shown in Table 3:

TABLE 3
Response to iodine vapor in cohort 3.

| | Visit 1 | Visit 2 | Visit 3 | Visit 4 | Visit5 |
|---|---|---|---|---|---|
| temp decline F. | 0 | 0.9 | 1.4 | 2.0 | 2.7 |
| oxygen increase | 0 | 1.0 | 1.4 | 2.5 | 3.4 |
| Average temp F. | 101.1 | 100.2 | 99.6 | 99.0 | 98.4 |
| Average oxygen | | | | | |
| saturation | 94.1 | 95.0 | 95.5 | 96.6 | 97.4 |
| (fingertips) | | | | | |

The improvement in temperature is similar, but the oxygen saturation improves more rapidly. It is likely that oxygen saturation is the quickest indication of a response to the treatment, and suggests that increasing the pH may have a role in helping the sicker patients quicker. Note that in cohort 3 the patients started off in worse condition, as evidenced by lower oxygen saturation, a critical determinant of COVID-19 patient wellness (Sherlaw-Johnson et al. [The impact of remote home monitoring of people with COVID-19 using pulse oximetry: A national population and observational study. E Clinical Medicine. 2022 March; 45:101318. doi: 10.1016/j.eclinm.2022.101318] and Jacob et al. [Prediction of COVID-19 deterioration in high-risk patients at diagnosis: an early warning score for advanced COVID-19 developed by machine learning. Infection. 2022 April; 50(2):359-370. Doi: 10.1007/s15010-021-01656-z].)

TABLE 4
A summary of the results from cohorts 2-3.

| | Visit 1 | Visit 2 | Visit 3 | Visit 4 | Visit 5 |
|---|---|---|---|---|---|
| oxygen increase | 0 | −0.7 | −0.6 | −0.8 | −1.1 |
| (control) | | | | | |
| Oxygen increase | 0 | 0.6 | 1.45 | 2 | 2.8 |
| cohort 2 (pH = 7) | | | | | |
| Oxygen increase | 0 | 1.0 | 1.4 | 2.5 | 3.4 |
| cohort 3 (pH = 8) | | | | | |

1. A method for treating a subject infected with a respiratory pathogen, comprising:
(a) warming an aqueous solution having a pH of 7.0-10.0, said solution comprising an iodine-releasing ionic compound and elemental iodine in a total iodine concentration of 0.5-10 ppm, to a temperature of 30-80 degrees centigrade (° C.), thereby generating a vapor; and
(b) administering said vapor to said subject's respiratory tract for 2 hours or less per day,
wherein total iodine delivered to said respiratory tract does not exceed 8 mg/kg of body weight/day.

2. A system for treating a subject infected with a respiratory pathogen, the system comprising:
a container with an aqueous solution having a pH of 7.0-10.0 disposed therein, said aqueous solution comprising an iodine-releasing ionic compound and elemental iodine in a total iodine concentration of 0.5-10 ppm;
a heating element configured for warming said aqueous solution in said container to a temperature of 30-80 degrees centigrade (° C.), thereby generating a vapor; and
a ventilator configured to ventilate said vapor such that said vapor reaches a respiratory tract of a subject infected with a respiratory pathogen;
wherein said ventilator is configured for administration of said vapor for 2 hours or less per day, wherein total iodine delivered to said respiratory tract does not exceed 8 mg/kg of body weight/day.

3. A measured dosage pack, comprising elemental iodine and an iodine-releasing ionic compound, accompanied by instructions for (a) introducing said dosage pack into an aqueous solution, having a pH of 7.0-10.0, in a total iodine concentration of 0.5-10 ppm; (b) warming said aqueous solution to a temperature of 30-80 degrees centigrade (° C.), thereby generating a vapor; and (c) administering said vapor to a respiratory tract of a subject infected with a respiratory pathogen, wherein said administration is for 2 hours or less per day, wherein total iodine delivered to said respiratory tract does not exceed 8 mg/kg of body weight/day.

4. An article of manufacture, comprising the system of claim 2, and instructions for use of said system in administering a vapor to a respiratory tract of a subject infected with a respiratory pathogen.

5. The method of claim 1, wherein said iodine is supplied to a lower respiratory tract of said subject.

6. The method of claim 1, wherein said respiratory pathogen is selected from the group consisting of viruses, bacteria, fungi, and mycobacteria.

7. The method of claim 6, wherein said respiratory pathogen is a virus.

8. The method of claim 7, wherein said respiratory pathogen is COVID-19.

9. The method of claim 1, wherein said iodine-releasing ionic compound is potassium iodide or povidone iodine.

10. The method of claim 1, wherein said vapor is administered simultaneously with an agent selected from an ACE inhibitor, vitamin D, hydroxychloroquine, zinc, a corticosteroid, and anti-clotting agent, a mouthwash, ivermectin, or a drug that increases intracellular pH or lysosomal pH in cells of said subject.

11. The method of claim 1, wherein said vapor and said subject are disposed within a confined space.

12. The method of claim 1, wherein said vapor is disposed within a breathing tube.

13. The method of claim 1, wherein said vapor is disposed within a respiratory face mask.

14. The method of claim 1, wherein said vapor is disposed within nasal prongs.

15. The method of claim 1, wherein said vapor is disposed within a ventilator.

16. The method of claim 1, wherein said vapor is nebulized.

17. The system of claim 2, wherein said ventilator is operatively associated with a solution container comprising said aqueous solution, and a heating element that facilitates vaporization of said aqueous solution.

18. The system of claim 2, wherein pressure and cadence of said ventilator are configured to enable deep breaths in said subject.

19. The system of claim 2, wherein said ventilator comprises an iodine autofill system.

20. The system of claim 19, wherein said ventilator further comprises an alkali autofill system.

21. The system of claim 2, wherein said ventilator comprises an alkali autofill system.
